(19) United States
(12) Patent Application Publication
(10) Pub. No.: US 2024/0236583 A1
(43) Pub. Date: Jul. 11, 2024

(54) MOLDED CAVITY FOR BEEPER RESONATION

(71) Applicant: Cardiac Pacemakers, Inc., St. Paul, MN (US)

(72) Inventors: Benjamin J. Haasl, Forest Lake, MN (US); James Michael English, Cahir (IE)

(21) Appl. No.: 18/409,223

(22) Filed: Jan. 10, 2024

Related U.S. Application Data
(60) Provisional application No. 63/438,306, filed on Jan. 11, 2023.

(51) Int. Cl. H04R 17/00

(52) U.S. Cl. CPC H04R 17/00

(57) ABSTRACT

An implantable medical device includes a metal case, a mold layer internal to the metal case and including a molded cavity arranged next to the metal case, and a piezo speaker arranged between the metal case and the molded cavity of the mold layer. The piezo speaker contacts the metal case.

### CLAIM OF PRIORITY

This application claims the benefit of U.S. Provisional Application No. 63/438,306 filed on Jan. 11, 2023, which is hereby incorporated by reference in its entirety.

### TECHNICAL FIELD

This patent application relates to implantable medical devices, and in particular to audio mechanisms for the devices.

### BACKGROUND

Implantable medical devices generally include a hermetically sealed metal case or metal housing. For implantable cardiac rhythm management devices, it is desired to sometimes bring the attention of the patient to the medical device. However, the metal case of the device is sealed from ambient air and is typically covered in body tissue. This makes it challenging to provide audio alerts that can be reliably heard by the patient.

### SUMMARY

Systems and methods are disclosed to audio mechanisms for medical devices. Example 1 includes subject matter (such as an implantable medical device) including a metal case, a mold layer internal to the metal case and including a molded cavity arranged next to the metal case, and a piezo speaker arranged between the metal case and the molded cavity of the mold layer. The piezo speaker contacts the metal case.

In Example 2, the subject matter of Example 1 optionally includes the molded cavity of the mold layer including a cavity surface opposite the piezo speaker and the cavity surface is concave.

In Example 3, the subject matter of one or both of Examples 1 and 2 optionally includes a mold layer including a ridge around the molded cavity and the piezo speaker contacts the ridge.

In Example 4, the subject matter of Example 3 optionally includes an insulating layer around the piezo speaker except for a portion of the piezo speaker that contacts the ridge.

In Example 5, the subject matter of one or both of Examples 3 and 4 optionally includes the molded cavity being a first molded circular cavity of the mold layer, and the mold layer including a second molded ring cavity having a ring shape. The ridge has a ring shape and is arranged between the first molded circular cavity and the second ring cavity; wherein the second molded ring cavity includes an insulating layer.

In Example 6, the subject matter of one or any combination of Examples 3-5 optionally includes the mold layer including a ridge around the molded cavity, and the molded cavity includes a gas sealed by the ridge around the molded cavity.

In Example 7, the subject matter of one or any combination of Examples 1-6 optionally includes a piezo speaker that includes a piezo element contacting a metal substrate; and further including an insulating layer covering the metal substrate and the piezo element.

In Example 8, the subject matter of Example 7 optionally includes a metal substrate including a brass disk, the piezo element includes a piezoelectric ceramic, and the insulation layer includes polyimide.

Example 9 includes subject matter (such as a method of forming an audio mechanism of an implantable medical device) or can optionally be combined with one or any combination of Examples 1-8 to include such subject matter, including forming a metal case, forming a mold layer including a molded cavity, arranging the mold layer in the metal case with the molded cavity arranged adjacent to a wall of the metal case, and arranging a piezo speaker between the metal case and the molded cavity of the mold layer such that the piezo speaker contacts the metal case.

In Example 10, the subject matter of Example 9 optionally includes forming a mold layer that includes a molded cavity having a concave cavity surface; and arranging the piezo speaker opposite the concave cavity surface and in contact with a metal wall of the metal case.

In Example 11, the subject matter of one or both of Examples 9 and 10 optionally includes forming a mold layer that includes a ridge around the molded cavity; and arranging the piezo speaker to contact the ridge of the mold layer and a metal wall of the metal case.

In Example 12, the subject matter of Example 11 optionally includes disposing an insulating layer around the piezo speaker except for a portion of the piezo speaker that contacts the ridge of the mold layer.

In Example 13, the subject matter of one or both of Examples 11 and 12 optionally includes forming the molded cavity as a first molded circular cavity of the mold layer; forming a second molded ring cavity having a ring shape, wherein the second molded ring cavity is formed around the first molded circular cavity and separated from the first molded circular cavity by the ridge of the mold layer; and disposing an insulating layer in the second molded ring cavity.

In Example 14, the subject matter of one or any combination of Examples 11-13 optionally includes forming a mold layer that includes a ridge around the molded cavity; and disposing a gas in the molded cavity and sealing the gas in the molded cavity using the ridge around the molded cavity.

In Example 15, the subject matter of one or any combination of Examples 9-14 optionally includes arranging, between the metal case and the molded cavity, a piezo speaker that includes a piezo element contacting a metal substrate; and covering the metal substrate and the piezo element with an insulating layer.

In Example 16, the subject matter of one or any combination of Examples 9-15 optionally includes arranging, between the metal case and the molded cavity, a piezo speaker that includes a circular piezoelectric ceramic contacting a brass disk; and covering the brass disk and the piezoelectric ceramic with a layer of polyimide′

Example 17 includes subject matter (such as an apparatus) or can optionally be combined with one or any combination of Examples 1-16 to include such subject matter, including a hermetically sealed metal case having a metal wall, a mold layer arranged within the metal case, the mold layer including a molded cavity and a ridge around the perimeter of the molded cavity, and a piezo circuit element including a piezoelectric ceramic. The ridge of the mold layer biases the piezo circuit element against the metal wall, and the piezoelectric ceramic is arranged within the molded cavity.

In Example 18, the subject matter of Example 17 optionally includes the molded cavity including a concave surface and a gas sealed in the molded cavity.

In Example 19, the subject matter of one or both of Examples 17 and 18 optionally includes a first insulating layer; and a piezo circuit element including the piezoelectric ceramic attached to a metal disk, and the insulating layer is arranged between the metal disk of the piezo circuit element and the metal wall.

In Example 20, the subject matter of Example 19 optionally includes a second insulating layer arranged between the piezo circuit element and the mold layer.

The non-limiting Examples can be combined in any permutation or combination. This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 is an illustration of portions of a system that uses a medical device.

FIG. 2 is a view of another example of a medical device.

FIG. 3 is another view of the medical device of FIG. 2.

FIG. 4 is an example of a piezo speaker.

FIG. 5 is another view of the medical device of FIG. 2 showing a mold layer internal to the medical device.

FIG. 6 is a cutaway view of a portion of an example of the mold layer of FIG. 5.

FIG. 7 is a cross-section view of a portion of an example of the mold layer of FIG. 5.

FIGS. 8 and 9 are cutaway views of a portion of an example of the mold layer and a piezo speaker.

FIG. 10 is a flow diagram of an example of a method of manufacture for an audio mechanism of a medical device.

### DETAILED DESCRIPTION

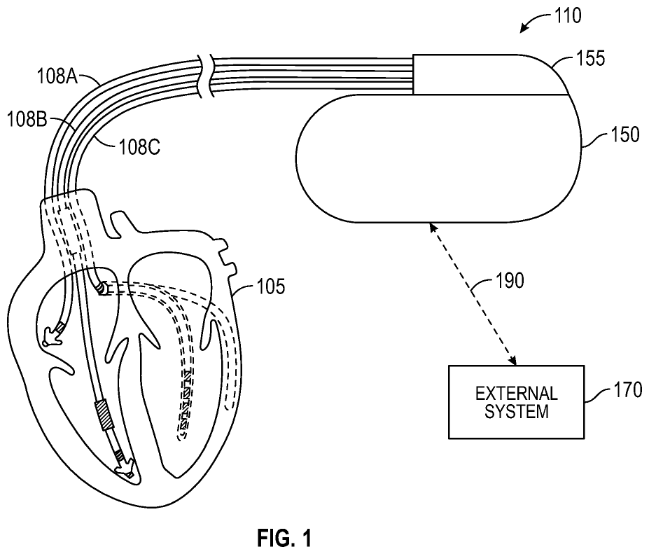

This document relates to an audio mechanism for an ambulatory medical device. FIG. 1 is an illustration of portions of a system that uses an implantable medical device (IMD). Some examples of the IMD 110 include a pacemaker, a defibrillator, a cardiac resynchronization therapy (CRT) device, a combination of such devices, or a diagnostic-only device. The system also typically includes an IMD programmer or other external device 170 that communicates wireless signals 190 with the IMD 110, such as by using radio frequency (RF) or other telemetry signals.

The IMD 110 can be coupled by one or more conductive leads 108A-C to heart 105. The cardiac leads 108A-C in the example of FIG. 1 include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals. Sensed electrical cardiac signals can be sampled to create an electrogram. An electrogram can be analyzed by the IMD and/or can be stored in the IMD and later communicated to an external device where the sampled signals can be displayed for analysis.

The IMD 110 includes a hermetically-sealed metal housing or metal case 150 that houses a battery and electronic circuits, and a header connector 155. Conductive leads (e.g., cardiac leads 108A-C) are connected to the IMD through the header connector 155. The cardiac leads 108A-C include right atrial (RA) lead 108A, right ventricle (RV) lead 108B, and a third cardiac lead 108C for placement in a coronary vein lying epicardially on the left ventricle (LV) via the coronary vein.

Because implantable cardiac rhythm management devices can be important to the health of the patient, the devices can perform self-diagnostics to detect upcoming or existing issues with operation of the devices. It is desirable to include alert mechanisms with the devices to call attention to any detected issues. An audio alert can be useful because an implantable device is typically not visible to the user. Designing an audio alert mechanism for an implantable device is not straightforward because the device is typically placed beneath layers of body tissue and is not exposed to air. The audio alert mechanism should be reliably audible to the patient but should not significantly drain the battery of the implantable device when activated.

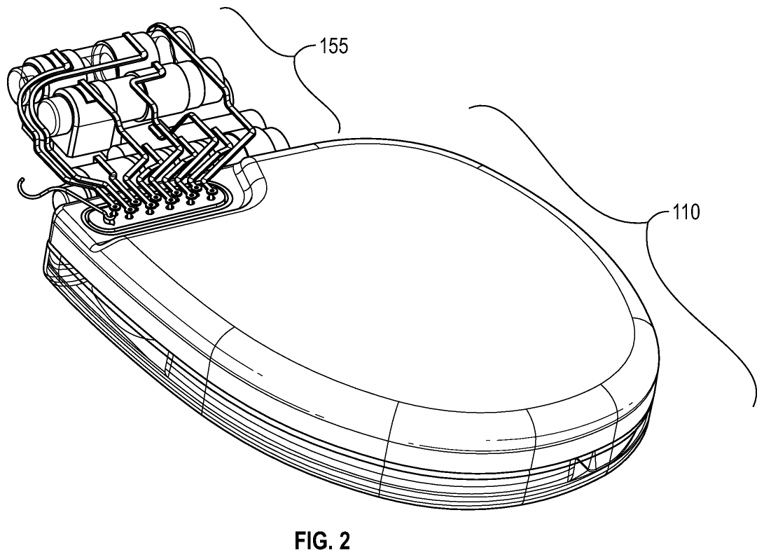

FIG. 2 is a view of another example of a medical device 110. The view shows the metal case 150 of the medical device and shows the header connector 155 without the mold material to expose the lead receptacles. The metal case can include titanium and is hermetically sealed (e.g., by welding).

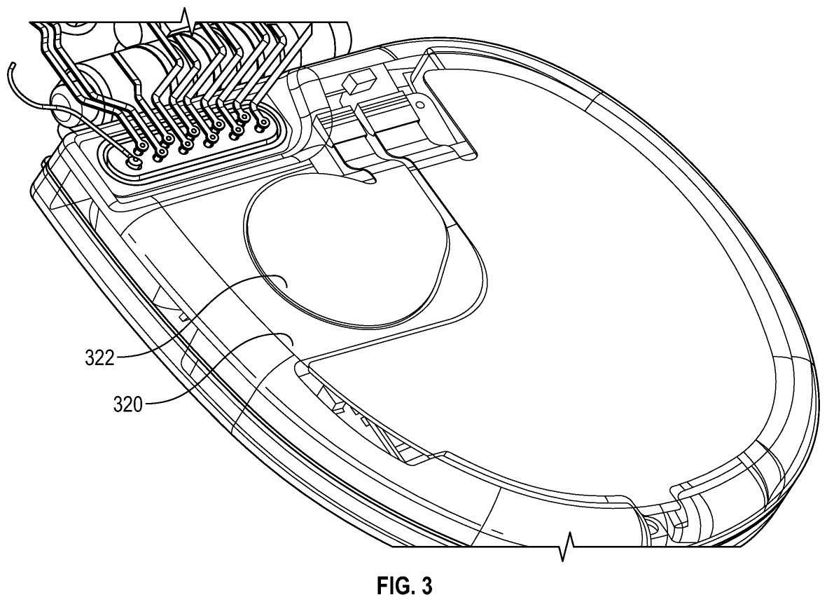

FIG. 3 is another view of the medical device 110 of FIG. 2. The top half of the metal case 150 is removed to show a mold layer 320. The mold layer can include molded plastic. The view also shows an insulating layer 322. The insulating layer 322 can include polyimide. Below the insulating layer 322 and above the mold layer 320 is a piezo speaker.

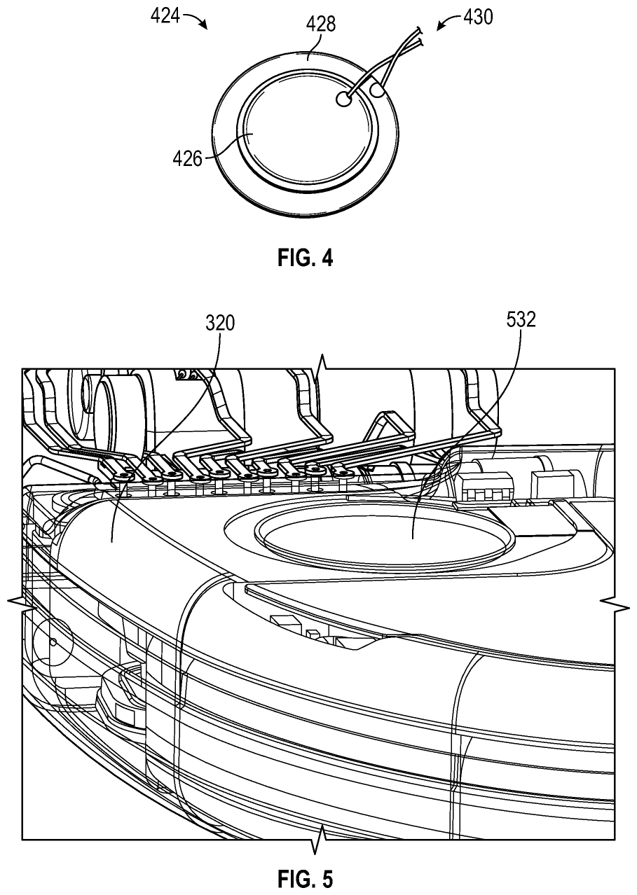

FIG. 4 is an example of a piezo speaker 424. It includes a piezoelectric element 426 (e.g., a piezoelectric ceramic) attached to a metal substrate 428 (e.g., a brass disk). Wires 430 are used to provide an electrical signal to the piezo speaker 424. The wires 430 may be metal traces included in a flexible printed ribbon connector. An alternating current (AC) signal can cause the piezoelectric element 426 to change shape and create sound waves for an audio alert mechanism. The piezo speaker 424 is positioned to contact the wall of the metal case. The insulating layer 322 electrically isolates the piezo speaker from the metal wall. Having the piezo speaker 424 against the metal wall causes the piezo speaker 424 to vibrate the metal wall when driven with an electrical signal. This increases the sound of an audio alert outside the hermetically sealed metal case 150.

FIG. 5 is another view of the medical device 110 of FIG. 2. The top half of the metal case 150 and the insulating layer 322 are removed to show more of the mold layer 320. The mold layer 320 includes a molded cavity 532 to provide a space in which the piezoelectric element 426 can mechanically resonate.

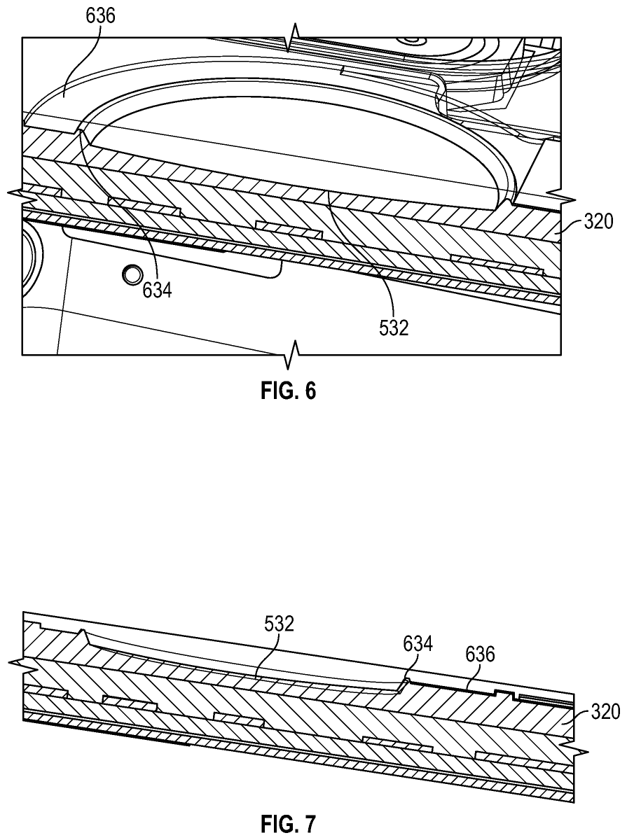

FIG. 6 is a cutaway view of a portion of an example of the mold layer 320 in the area of the molded cavity 532. The cutaway view shows that the bottom surface of the molded cavity 532 is not flat and is a concave surface. To improve the performance of the piezo speaker 424, the molded cavity 532 is sealed with a gas in the cavity. The molded cavity 532 is shaped so that a standing wave is formed in the molded cavity 532 when the piezo speaker 424 resonates. When the piezo speaker 424 resonates, part of the movement of the piezoelectric element 426 is toward the cavity bottom surface which compresses the gas within the cavity. When the piezoelectric element 426 moves in the other direction, the compressed gas provides extra force to drive the piezoelectric element 426 against the metal wall.

FIG. 6 also shows that the mold layer 320 includes a molded ridge 634 around the perimeter of the molded cavity 532. The piezo speaker 424 sits above the molded cavity 532 on the ridge 634. Mounting the piezo speaker 424 on a ridge minimizes the surface of the piezo speaker 424 that is dampened by the surface of the mold layer 320. The ridge 634 also biases the piezo speaker against the metal case and is used to seal the gas within the molded cavity 532.

In the example of FIG. 6, the molded cavity 532 has a circular shape and the molded ridge 634 is shaped as a ring around the circular cavity. The cavity may have other shapes (e.g., a square shape). The mold layer 320 also includes a second molded ring cavity 636 around the ridge 634. The second cavity may include an insulating layer (e.g., polyimide tape). FIG. 7 is a cross-section view of a portion of the example of the mold layer 320 in FIG. 6. The cross-section view shows the concave cavity 532, the ridge 634 and the second cavity 636.

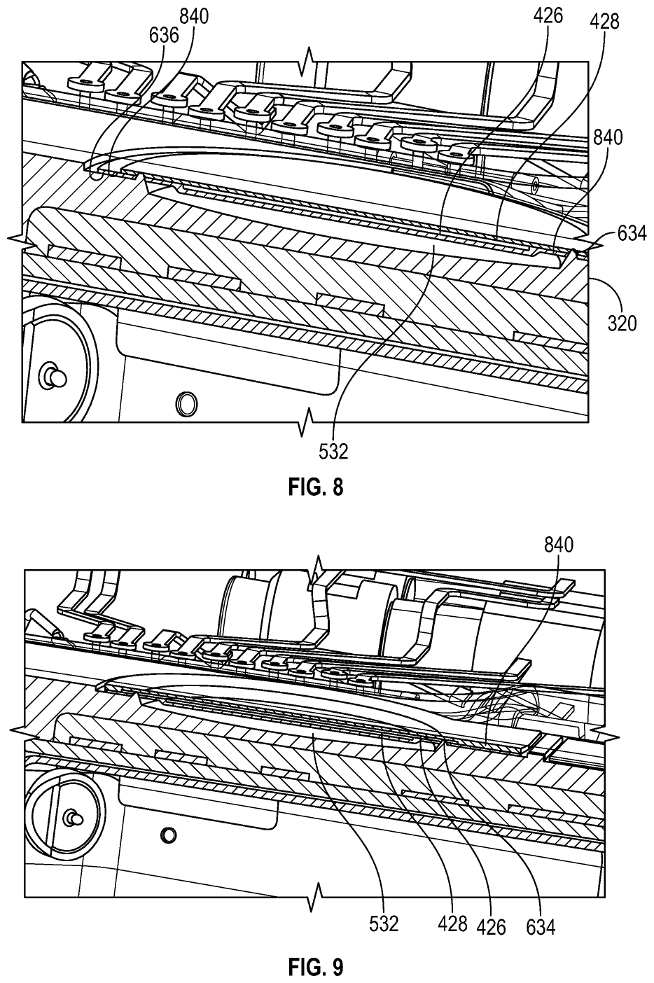

FIG. 8 is a cutaway view of a portion of an example of the mold layer 320, molded cavity 532, ridge 634, piezoelectric element 426, and metal substrate 428 of the piezo speaker 424. An insulation layer 840 covers the bottom side of the piezo speaker 424. In some examples, the insulation layer 840 includes polyimide. The insulation layer 840 is disposed in the molded ring cavity 636 around the ridge. In some examples, the insulation layer 840 does not cover the ridge 634.

FIG. 9 is a cutaway view of a portion of another example of the mold layer 320, molded cavity 532, ridge 634, piezoelectric element 426, metal substrate 428 of the piezo speaker 424, and the insulation layer 840. The insulation layer 840 covers both the top side and the bottom side of the piezo speaker 424. The insulation layer 840 may insulate metal traces that carry electricity to the piezo speaker 424 and may insulate other metal traces from the metal substrate 428.

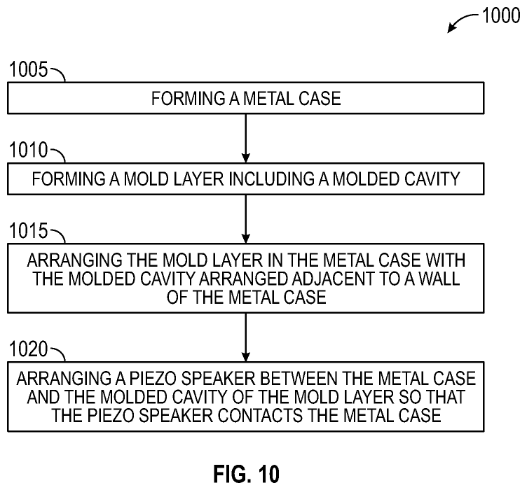

FIG. 10 is a flow diagram of an example of a method 1000 of manufacture for an audio mechanism of a medical device. At block 1005, a metal case is formed, such as the metal case 150 in FIG. 2 for example. At block 1010 a mold layer is formed, such as the mold layer 320 in FIG. 3. for example. The mold layer 320 may include molded plastic. The mold layer 320 includes a molded cavity such as the molded cavity 532 of FIG. 5. In some examples, forming the mold layer 320 includes forming a molded cavity 532 that has a concave cavity surface. In some examples, the mold layer 320 includes a ridge around the molded cavity (e.g., molded ridge 634 of FIG. 6). In some examples, the mold layer 320 includes a second cavity (e.g., cavity 636 in FIG. 6) around the first molded circular cavity and separated from the first molded circular cavity by the ridge. The second cavity 636 may be less deep than the first cavity.

At block 1015, the mold layer 320 is arranged in the metal case 150 with the molded cavity 532 arranged adjacent to a wall of the metal case 150. At block 1020, a piezo speaker (e.g., piezo speaker 424 of FIG. 4) is arranged between the metal case and the molded cavity of the mold layer so that the piezo speaker 424 contacts the metal case 150. The piezo speaker 424 can be positioned to contact the ridge 634 of the mold layer 320 and a metal wall of the metal case 150.

In some examples, the method 1000 includes disposing a gas in the molded cavity next to the piezo speaker 424 and sealing the gas in the molded cavity using the ridge around the molded cavity. In some examples, the method 1000 includes disposing an insulating layer around the piezo speaker 424. In some examples, the method 1000 includes disposing an insulating layer around the piezo speaker 424 including, including insulation in the second cavity 636 and excluding insulation on the ridge 634.

The techniques described herein provide an audio alert mechanism for a medical device. The techniques include structural features to make the audio alert mechanism reliably audible to the patient and to reduce drain on the battery when the audio mechanism is activated.

### ADDITIONAL DESCRIPTION

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An implantable medical device, the device comprising:
a metal case;
a mold layer internal to the metal case and including a molded cavity arranged next to the metal case; and
a piezo speaker arranged between the metal case and the molded cavity of the mold layer, wherein the piezo speaker contacts the metal case.

2. The device of claim 1, wherein the molded cavity of the mold layer includes a cavity surface opposite the piezo speaker and the cavity surface is concave.

3. The device of claim 1, wherein the mold layer includes a ridge around the molded cavity and the piezo speaker contacts the ridge.

4. The device of claim 3, including an insulating layer around the piezo speaker except for a portion of the piezo speaker that contacts the ridge.

5. The device of claim 3,
wherein the molded cavity is a first molded circular cavity of the mold layer, and the mold layer includes a second molded ring cavity having a ring shape;
wherein the ridge has a ring shape and is arranged between the first molded circular cavity and the second ring cavity; and
wherein the second molded ring cavity includes an insulating layer.

6. The device of claim 1, wherein the mold layer includes a ridge around the molded cavity, and the molded cavity includes a gas sealed by the ridge around the molded cavity.

7. The device of claim 1,
wherein the piezo speaker includes a piezo element contacting a metal substrate; and
wherein the device further includes an insulating layer covering the metal substrate and the piezo element.

8. The device of claim 7, wherein the metal substrate includes a brass disk, the piezo element includes a piezoelectric ceramic, and the insulation layer includes polyimide.

9. A method of forming an audio mechanism of an implantable medical device, the method comprising:
forming a metal case;
forming a mold layer including a molded cavity;
arranging the mold layer in the metal case with the molded cavity arranged adjacent to a wall of the metal case; and
arranging a piezo speaker between the metal case and the molded cavity of the mold layer, wherein the piezo speaker contacts the metal case.

10. The method of claim 9,
wherein the forming the mold layer includes forming a mold layer that includes a molded cavity having a concave cavity surface; and
wherein arranging the piezo speaker includes arranging the piezo speaker opposite the concave cavity surface and in contact with a metal wall of the metal case.

11. The method of claim 9,
wherein the forming the mold layer includes forming a mold layer that includes a ridge around the molded cavity; and
wherein the arranging the piezo speaker includes arranging the piezo speaker to contact the ridge of the mold layer and a metal wall of the metal case.

12. The method of claim 11, including disposing an insulating layer around the piezo speaker except for a portion of the piezo speaker that contacts the ridge of the mold layer.

13. The method of claim 11, wherein the forming the mold layer includes:
forming the molded cavity as a first molded circular cavity of the mold layer;
forming a second molded ring cavity having a ring shape, wherein the second molded ring cavity is formed around the first molded circular cavity and separated from the first molded circular cavity by the ridge of the mold layer; and
disposing an insulating layer in the second molded ring cavity.

14. The method of claim 9,
wherein the forming the mold layer includes forming a mold layer that includes a ridge around the molded cavity; and
wherein the method further includes disposing a gas in the molded cavity and sealing the gas in the molded cavity using the ridge around the molded cavity.

15. The method of claim 9, wherein the arranging the piezo speaker includes:
arranging, between the metal case and the molded cavity, a piezo speaker that includes a piezo element contacting a metal substrate; and
covering the metal substrate and the piezo element with an insulating layer.

16. The method of claim 9, wherein the arranging the piezo speaker includes:
arranging, between the metal case and the molded cavity, a piezo speaker that includes a circular piezoelectric ceramic contacting a brass disk; and
covering the brass disk and the piezoelectric ceramic with a layer of polyimide.

17. An apparatus comprising:
a hermetically sealed metal case having a metal wall;
a mold layer arranged within the metal case, the mold layer including a molded cavity and a ridge around the perimeter of the molded cavity; and
a piezo circuit element including a piezoelectric ceramic, wherein the ridge of the mold layer biases the piezo circuit element against the metal wall, and the piezoelectric ceramic is arranged within the molded cavity.

18. The apparatus of claim 17, wherein the molded cavity includes a concave surface and a gas sealed in the molded cavity.

19. The apparatus of claim 17, including:
a first insulating layer; and
wherein the piezo circuit element includes the piezoelectric ceramic attached to a metal disk, and the insulating layer is arranged between the metal disk of the piezo circuit element and the metal wall.

20. The apparatus of claim 19, including a second insulating layer arranged between the piezo circuit element and the mold layer.
